(19) United States
(12) Patent Application Publication
(10) Pub. No.: US 2026/0340241 A1
(43) Pub. Date: Oct. 29, 2026

(54) Myocardial Blood Flow Simulation

(71) Applicant: Siemens Healthineers AG, Forchheim (DE)

(72) Inventor: Xi Zhao, Shanghai (CN)

(73) Assignee: Siemens Healthineers AG, Forchheim (DE)

(21) Appl. No.: 19/658,789

(22) Filed: Apr. 27, 2026

(30) Foreign Application Priority Data
Apr. 27, 2025 (CN) 202510546723.X

(51) Int. Cl. G06F 30/28; A61B 6/50; G06F 113/08; G06T 7/00; G06T 7/11

(52) U.S. Cl. CPC G06F 30/28; G06T 7/0004; G06T 7/11; A61B 6/504; G06F 2113/08; G06T 2207/30048; G06T 2207/30104

(57) ABSTRACT

The disclosure relates to a method and apparatus for simulating myocardial blood flow, and a device. The method includes: acquiring an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery; for the at least one first region, acquiring a first time-density curve to obtain a maximum slope of the first time-density curve; for the at least one second region, acquiring a second time-density curve to obtain a maximum enhancement value of the second time-density curve; and determining the myocardial blood flow according to the maximum slope and the maximum enhancement value.

### TECHNICAL FIELD

The present disclosure relates to the technical field of CT imaging, and, in particular, to a method for simulating myocardial blood flow, an apparatus for simulating myocardial blood flow, and an electronic device.

### BACKGROUND

Coronary heart disease is one of the highest mortality diseases in the world. Cardiovascular disease has become a major public health problem in recent years, with the continued rise in the incidence and mortality of coronary heart disease. Coronary tomography angiography (CTA) has been widely used as a noninvasive imaging technique for assessing the degree of coronary artery stenosis and the efficacy of treatment in patients with coronary artery disease. However, it cannot provide accurate quantitative information on coronary hemodynamics and myocardial blood flow (MBF). Therefore, methods such as myocardial CT perfusion (CTP) are still required to measure myocardial blood flow and myocardial activity.

However, the CTP uses a higher dose of the contrast agent, which increases radiation exposure and poses a higher health risk. In addition, CTP examinations are time-consuming.

Therefore, there is an urgent need for a method for simulating myocardial blood flow that reduces patient health risks, reduces medical costs, and improves the efficiency of obtaining myocardial blood flow.

### SUMMARY

In view of this, according to an aspect of the present disclosure, there is provided a method for simulating myocardial blood flow, comprising: acquiring an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery; for the at least one first region, acquiring a first time-density curve to obtain a maximum slope of the first time-density curve; for the at least one second region, acquiring a second time-density curve to obtain a maximum enhancement value of the second time-density curve; and determining the myocardial blood flow according to the maximum slope and the maximum enhancement value.

According to another aspect of the present disclosure, there is provided an apparatus for simulating myocardial blood flow, the apparatus comprising: a first unit, configured to acquire an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery; a second unit, configured to, for the at least one first region, acquire a first time-density curve to obtain a maximum slope of the first time-density curve; a third unit, configured to, for the at least one second region, acquire a second time-density curve to obtain a maximum enhancement value of the second time-density curve; and a fourth unit, configured to determine the myocardial blood flow according to the maximum slope and the maximum enhancement value.

According to still another aspect of the present disclosure, there is provided an electronic device, comprising: at least one processor; and at least one memory in communicative connection with the at least one processor, wherein the at least one memory stores instructions which are executable by the at least one processor, and the instructions are executed by the at least one processor, to enable the at least one processor to perform the method for simulating myocardial blood flow.

It should be understood that the content described in this section is not intended to identify key or important features of aspects of the present disclosure and is not intended to limit the scope of the present disclosure. Other features of the present disclosure will become easy to understand through the following specification.

### BRIEF DESCRIPTION OF THE DRAWINGS

The drawings, which illustratively show aspects and form part of this specification, are used together with the textual description of this specification to explain exemplary ways of implementing aspects. The aspects shown are provided for exemplary purposes only, and do not limit the scope of the claims. In all of the drawings, identical reference signs denote similar but not necessarily identical key elements.

The aspects of the present disclosure will be described in detail below with reference to the drawings, to give those skilled in the art a clearer understanding of the above-mentioned and other features and advantages of the present disclosure. In the drawings:

FIG. 1 shows a flowchart of a method for simulating myocardial blood flow according to an aspect of the present disclosure;

FIG. 2 shows a flowchart of a partial process of the method for simulating myocardial blood flow in FIG. 1;

FIG. 3 shows a flowchart of a partial process of the method for simulating myocardial blood flow in FIG. 1;

FIG. 4 shows a flowchart of a treatment plan recommendation method according to an aspect of the present disclosure;

FIG. 5 shows a schematic diagram of an angiographic image generated by using the method for simulating myocardial blood flow according to the aspect of the present disclosure;

FIGS. 6A and 6B show schematic diagrams of perfusion images of myocardial blood flow acquired by using the method for simulating myocardial blood flow according to the aspect of the present disclosure;

FIG. 7 shows a block diagram of the structure of an apparatus for simulating myocardial blood flow according to an aspect of the present disclosure; and

FIG. 8 shows a block diagram of an exemplary electronic device according to an aspect of the present disclosure.

### DETAILED DESCRIPTION

Illustrative aspects of the present disclosure will be described below with reference to the drawings, including various details of the aspects of the present disclosure to aid understanding, and these should be regarded as merely illustrative. Therefore, it should be appreciated by those skilled in the art that various changes and modifications could be made to the aspects described here without deviating from the scope of the present disclosure. In addition, for clarity and conciseness, descriptions of commonly known functions and structures are omitted in the following descriptions.

In the present disclosure, unless otherwise stated, the use of the terms “first”, “second”, etc. to describe various key elements is not intended to define a positional relationship, time sequence relationship, or importance relationship between these key elements; such terms are merely used to distinguish one element from another. In some examples, a first key element and a second key element may refer to the same instance of the described element, whereas in some cases, based on the description in the context, they may also refer to different instances.

Terms used in the descriptions of the various examples in the present disclosure are merely intended to describe specific examples, and are not intended to impose restrictions. Unless clearly indicated otherwise in the context, if the quantity of a key element is not specifically defined, the key element may be one or more. In addition, the term “and/or” used in the present disclosure covers any one of the listed items and all possible combinations.

As described above, a professional can assess the blood flow function of a patient by using invasive coronary tomography angiography or myocardial CT perfusion to obtain information about the patient's myocardial blood flow. In some scenarios, the patient is not eligible for either of these examinations. For example, the patient's physical condition does not allow the injection of a high dose of contrast agent or invasive examinations, and the patient's condition is urgent, making it impossible to wait for perfusion imaging results. In addition, the two examination methods described above do not accurately reflect early or microvascular ischemia.

Based on this, the present disclosure provides a method for simulating myocardial blood flow, a treatment plan recommendation method, an apparatus for simulating myocardial blood flow, and an electronic device.

The aspects of the present disclosure will be described in detail below with reference to the drawings.

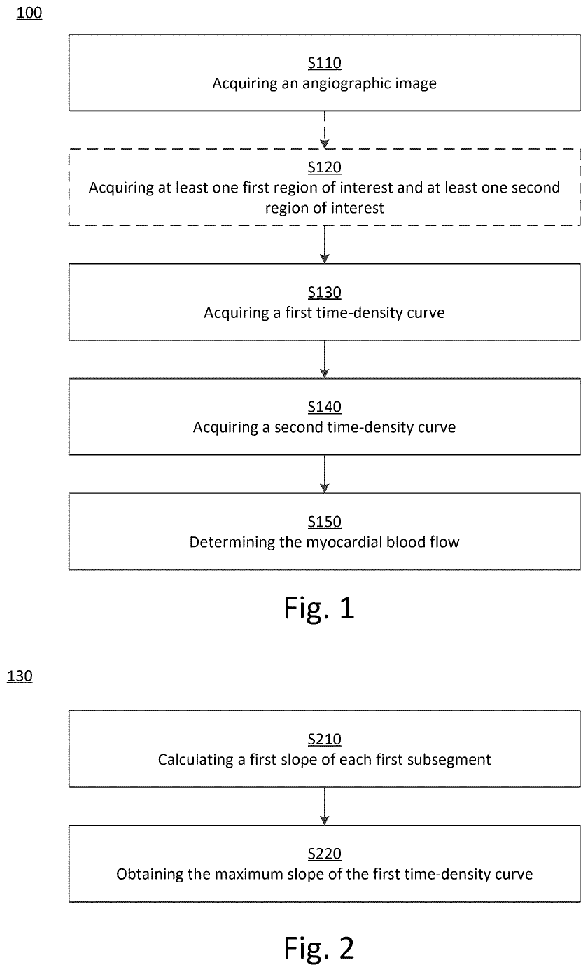

FIG. 1 shows a flowchart of a method 100 for simulating myocardial blood flow according to an aspect of the present disclosure.

Referring to FIG. 1, the method 100 for simulating the myocardial blood flow comprises:
step S110: acquiring an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery;
step S130: for the at least one first region, acquiring a first time-density curve to obtain a maximum slope of the first time-density curve;
step S140: for the at least one second region, acquiring a second time-density curve to obtain a maximum enhancement value of the second time-density curve; and
step S150: determining the myocardial blood flow according to the maximum slope and the maximum enhancement value.

The method 100 provides a method for simulating myocardial blood flow. Specifically, the method 100 is to simulate myocardial blood flow by acquiring time-density curves from specific regions of the CT angiographic image of the coronary artery.

The time-density curve (TDC) is a graph that reflects the variation of CT values over time in a specific region of a patient (such as a blood vessel or tissue). It typically uses time as the horizontal axis and CT values as the vertical axis, and is used to reflect the density changes in the specific region at different time points. For example, in the myocardial CT perfusion, discrete CT value data at a plurality of time points is acquired and fitted into a time-density curve. Still taking the myocardial CT perfusion as an example, to further calculate myocardial blood flow, both the time-density curve for the lumen region of the coronary artery of the patient and the time-density curve for the myocardial region of the left ventricle of the patient need to be measured. The two time-density curves each need to completely include the entire process in which the CT value rises to a peak and then decreases to a stable level. Further, the myocardial blood flow can be calculated from the maximum slope of the time-density curve for the myocardial region of the left ventricle and the maximum enhancement value of the time-density curve for the lumen region of the coronary artery.

In the example of step S110, similar to the myocardial CT perfusion as described above, the method 100 also requires the simultaneous acquisition of an angiographic image that includes at least one myocardial region located in the left ventricle and at least one lumen region located in the coronary artery.

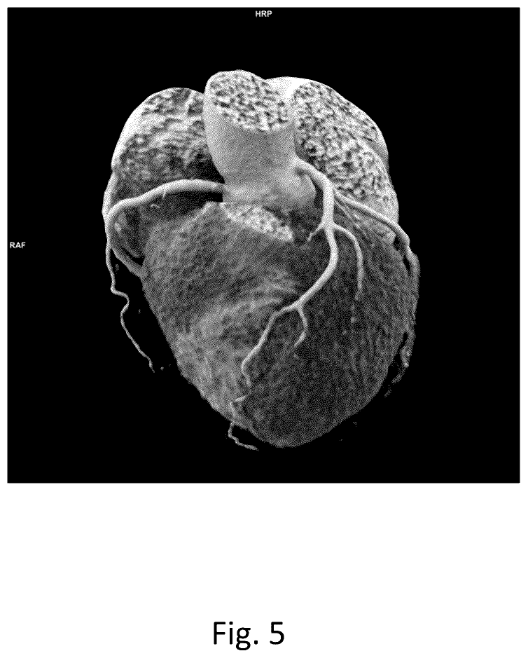

FIG. 5 shows a schematic diagram of an angiographic image generated by using the method for simulating myocardial blood flow according to the aspect of the present disclosure. As shown in FIG. 5, the angiographic image obtained by the method 100 may be a three-dimensional reconstructed image including the myocardial region of the left ventricle and the lumen region of the coronary artery.

According to some aspects of the present disclosure, after step S110, the method may further comprise step S120: acquiring at least one first region of interest and at least one second region of interest on the basis of the angiographic image.

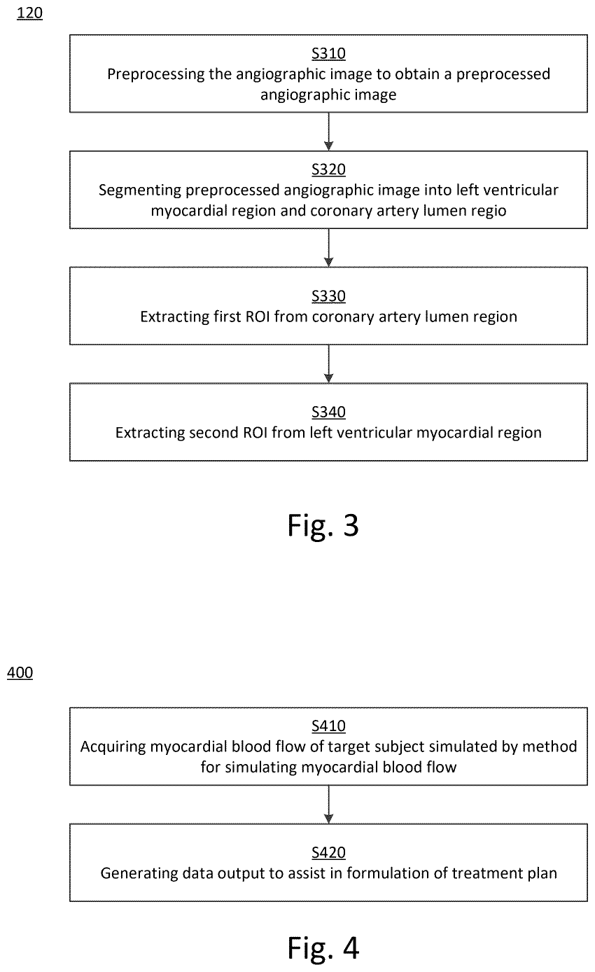

FIG. 3 shows a flowchart of a partial process of the method for simulating myocardial blood flow in FIG. 1. As shown in FIG. 3, step S120 further comprises:
step S310: preprocessing the angiographic image to obtain a preprocessed angiographic image;
step S320: dividing the preprocessed angiographic image into the myocardial region of the left ventricle and the lumen region of the coronary artery;
step S330: in the myocardial region of the left ventricle, extracting at least one first region of interest as the at least one first region; and
step S340: in the lumen region of the coronary artery, extracting at least one second region of interest as the at least one second region.

In the example of step S310, the preprocessing of the angiographic image includes noise reduction, such as Gaussian filtering, median filtering, image registration, resampling, normalization processing, etc. With the preprocessing method described above, the image quality of the angiographic image can be improved, thereby providing a reliable basis for the subsequent calculation of myocardial blood flow.

Based on this, in the example of step S320, the preprocessed angiographic image is divided into the myocardial region of the left ventricle and the lumen region of the coronary artery, making it convenient to separately obtain the time-density curve for the lumen region of the coronary artery of the patient and the time-density curve for the myocardial region of the left ventricle of the patient.

Further, in the example of steps S330 and S340, regions of interest (ROI) are respectively extracted from the lumen region of the coronary artery and the myocardial region of the left ventricle to determine the first region and the second region. The region of interest may be automatically identified or delineated by a professional based on clinical experience and relevant knowledge. The purposes of doing so are as follows: First, delineating the ROI reduces unnecessary computation and improves computational efficiency, especially when processing a large volume of data, which can save time and computational resources. Second, delineating the ROI can exclude interference from irrelevant regions and improve diagnostic accuracy. Third, delineating the ROI helps standardize the analysis process, ensuring comparability of comparisons between different time points or different patients. Finally, in dynamic monitoring, delineating the ROI enables tracking of changes in specific regions, such as treatment effects or disease progression.

With step S120 described above, i.e., steps S310 to S340, the acquired angiographic image is preprocessed, thereby facilitating the accurate simulation of myocardial blood flow. In addition, extracting the region of interests and using the region of interests as the first region and the second region are conducive to the subsequent generation of time-density curves and simplify the calculation of myocardial blood flow.

With continued reference to FIG. 1, in the example of step S130, the first time-density curve for the first region is generated, and the maximum slope of the first time-density curve is calculated; and in the example of step S140, the second time-density curve for the second region is generated, and the maximum enhancement value for the second time-density curve is calculated.

As described above, the time-density curves in the myocardial CT perfusion each need to completely include the entire process in which the CT value rises to a peak and then decreases to a stable level; however, this results in a higher radiation dose and a longer data acquisition time. To solve this problem, the time-density curve in the aspects of the present disclosure involves sparse sampling; that is, in step S130, for the first region and the second region, data is acquired only for a specific time point or time period, thereby forming two “incomplete” time-density curves.

In some aspects of the present disclosure, the time from which the acquisition of the time-density curve starts may be set by setting a trigger value. Specifically, the acquisition of first time-density curve starts from a first trigger time, and the first trigger time is a time at which a first monitored CT value of at least one first region exceeds a preset first trigger value for the first time; and the acquisition of the second time-density curve starts from a second trigger time, and the second trigger time is a time at which a second monitored CT value of at least one second region exceeds a preset second trigger value for the first time. It is also possible to set the start of acquisition at a time point occurring a certain interval after the injection of the contrast agent.

Similarly, the time at which the acquisition of the time-density curve stops may be set by setting a threshold. Specifically, the acquisition of the first time-density curve stops at a first cutoff time, and the first cutoff time is configured as a time at which the first monitored CT value of at least one first region exceeds a preset first threshold for the first time; and the acquisition of the second time-density curve stops from a second cutoff time, and the second cutoff time is configured to be a time at which the second monitored CT value of at least one second region exceeds a preset second threshold for the first time. It is also possible to set the time at which the acquisition stops at a time point occurring a certain interval after the start of the acquisition.

FIG. 2 shows a flowchart of a partial process of the method for simulating myocardial blood flow in FIG. 1. As shown in FIG. 2, in step S130, the maximum slope of the first time-density curve is determined by means of the following steps:
S210: for a plurality of first subsegments of the first time-density curve, calculating a first slope of each first subsegment; and
S220: determining a maximum slope calculated from the plurality of first subsegments as the maximum slope, wherein the plurality of first subsegments are composed of lines connecting each two temporally adjacent first monitored CT values among a plurality of first monitored CT values of the at least one first region in an angiography process.

In the example of step S210, it can be seen that the first time-density curve is composed of first subsegments between multiple adjacent data points. Compared with the time-density curve fitted using dozens of data points in the conventional myocardial CT perfusion, the first time-density curve in the aspects of the present disclosure is directly composed of the plurality of first subsegments, such that the first slope of each first subsegment can be calculated simply and rapidly, which is conducive to improving the efficiency of simulating myocardial blood flow.

In the example of step S220, the maximum value among the first slopes of the plurality of first subsegments is used directly as the maximum slope, thereby quickly obtaining the maximum slope of the first time-density curve.

In addition, for step S140, the maximum enhancement value may be determined by calculating the difference between maximum and minimum values among a plurality of second monitored CT values of at least one second region in an angiography process, thereby simply calculating the maximum enhancement value.

Based on this, in the example of step S150, the myocardial blood flow of the coronary artery is determined according to the maximum slope described above and the maximum enhancement value described above.

The method in the aspects of the present disclosure simulates the myocardial blood flow of the coronary artery by means of data from CT angiography of the coronary artery and the left ventricle adjacent to the coronary artery, i.e., the angiographic image, the first time-density curve, and the second time-density curve. Therefore, the patient does not need to undergo invasive examinations or receive the injection of a large dose of contrast agent, thereby reducing associated health risks and complications. The method in the aspects of the present disclosure directly uses the maximum slope and the maximum enhancement value of the time-density curves, thereby enabling rapid provision of an estimated myocardial blood flow value, making it suitable for real-time clinical decision-making, and particularly advantageous for preliminary assessment of patients with acute coronary heart disease or myocardial ischemia in emergency situations. In addition, because coronary CT angiography has been widely used in clinical practice in the assessment of coronary artery disease, the method in the aspects of the present disclosure can be easily combined with existing clinical workflows, thereby reducing application costs and the difficulty of implementation.

In some aspects of the present disclosure, the myocardial blood flow is calculated by means of a weighted linear combination of the maximum slope and the maximum enhancement value. Compared with calculating the myocardial blood flow by using a complex hemodynamic model in the myocardial CT perfusion, the method in the aspects of the present disclosure greatly simplifies the calculation process through a linear formula including the maximum slope and the maximum enhancement value, thereby providing a relatively accurate preliminary estimate of myocardial blood flow.

Specifically, the following empirical formula may be used to calculate the myocardial blood flow:

MBF=(k₁×S+k₂×E)+b
where MBF is the myocardial blood flow, S is the maximum slope of the first time-density curve, and E is the maximum enhancement value of the second time-density curve; k₁, k₂, and b are coefficients determined according to clinical data or experiments, respectively; k1, k2, and b may be constants.

The linear empirical formula described above does not require significant computational resources or detailed anatomical and physiological information, and provides an objective and repeatable assessment of myocardial blood flow, thereby improving the accuracy and consistency of disease diagnosis based on myocardial blood flow, and reducing uncontrollable changes in clinical practice associated with subjective interpretation.

In some aspects of the present disclosure, the region of myocardial ischemia can be determined by the simulated myocardial blood flow. For example, the region of myocardial ischemia may be displayed in the acquired angiographic image using a color or CT value that differs from that in the normal myocardial region, or the region of myocardial ischemia may be marked with special marking symbols in the angiographic image. It is also possible to describe the region of myocardial ischemia, the degree and proportion of myocardial ischemia, and so on by means of text.

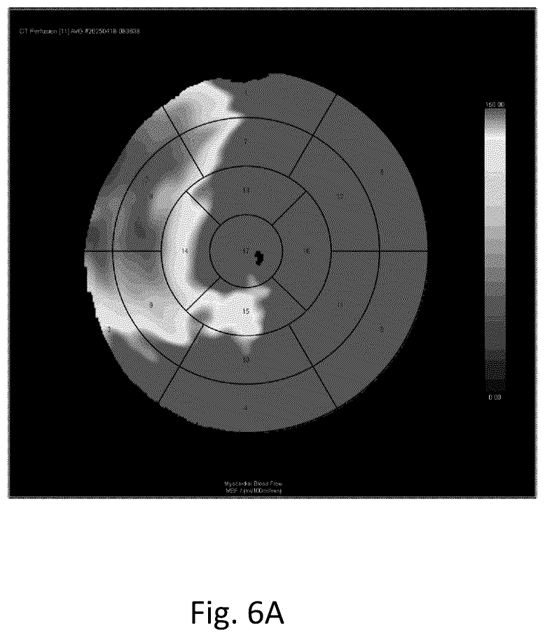

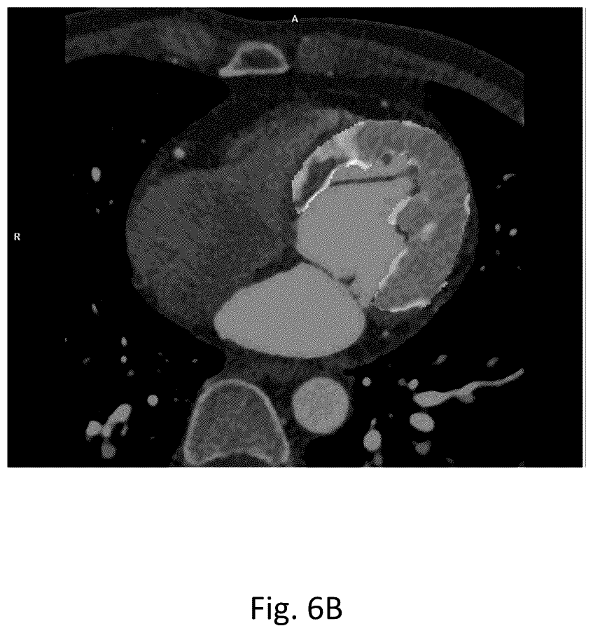

FIGS. 6A and 6B show schematic diagrams of perfusion images of myocardial blood flow acquired by using the method for simulating myocardial blood flow according to the aspect of the present disclosure. As shown in FIGS. 6A and 6B, in some aspects of the present disclosure herein, a visual map of the distribution of the coronary blood flow may be generated based on the simulated myocardial blood flow; and/or the angiographic image and the myocardial blood flow are fused to generate a myocardial perfusion image. Specifically, as shown in FIG. 6A, the visual map of the distribution of the coronary blood flow may be presented in the form of a bull's-eye diagram. In the bull-eye diagram, by using different colors or grayscale levels to represent the percentage change in myocardial wall thickness during different systolic and diastolic periods, myocardial perfusion, and dynamic behaviors of myocardial tissue such as velocity, displacement, strain, strain rate, and rotational angle, the severity and extent of myocardial damage in different cardiac segments can be visually, quickly, and accurately determined, thereby comprehensively evaluating global and regional changes in cardiac systolic and diastolic function.

The angiographic image may be fused with myocardial blood flow, as shown in FIG. 6B. The fused image of FIG. 6B is based on a two-dimensional tomographic image of the angiographic image and the myocardial blood flow at that location. It is also possible to use the three-dimensional reconstructed angiographic image as described above to fuse with the myocardial blood flow, thereby obtaining a dynamic myocardial perfusion image.

According to another aspect of the present disclosure, a treatment plan recommendation method is provided.

FIG. 4 shows a flowchart of a treatment plan recommendation method 400 according to an aspect of the present disclosure. As shown in FIG. 4, the treatment plan recommendation method 400 comprises:
step S410: acquiring myocardial blood flow of a target subject simulated by a method for simulating myocardial blood flow; and
step S420: based on the myocardial blood flow and individualized information of the target subject, generating data output to assist in the formulation of a treatment plan.

In the example of step S410, the myocardial blood flow of the target subject is acquired using the method 100 as described in FIG. 1. Thus, the operations, features, and advantages described above with respect to the method 100 are also applicable to the method 400. For the sake of conciseness, some operations, features, and advantages are not described again here.

In the example of step S420, the individualized information of the target subject may include basic information of the target subject, such as the height, weight, blood type, family history, allergies, etc. of the target subject; the individualized information of the target subject may also include pathological information of the target subject, such as ECG information, chest X-ray examination, etc. of the target subject; the individualized information of the target subject may further include subjective information of the target subject, for example, information such as treatment preferences of the target subject, treatment adherence of the target subject, and psychological states of the target subject.

A preliminary treatment plan may be automatically generated based on the individualized information and myocardial blood flow for the target subject described above. The treatment plan may include recommendations for the choice of therapy, such as which medications to use, whether to undergo surgery or interventional treatment, and other supportive therapies; the treatment plan may also include recommendations for lifestyle intervention, such as dietary adjustments, exercise plans, and smoking or alcohol cessation; the treatment plan may further include recommendations for psychological interventions to help the target subject cope with the psychological stress caused by the disease.

The above description does not involve the limitations of step S420, and depending on actual needs, the appropriate form and content can be selected to generate the data output for generating the treatment plan.

According to another aspect of the present disclosure, an apparatus for simulating myocardial blood flow is provided.

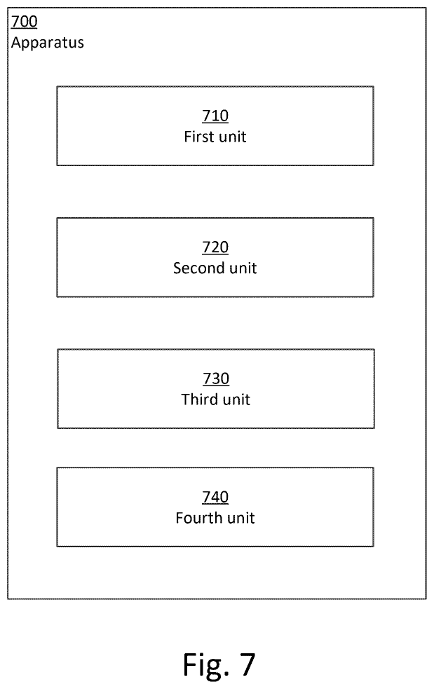

FIG. 7 shows a block diagram of the structure of an apparatus 700 for simulating myocardial blood flow according to an aspect of the present disclosure. As shown in FIG. 7, the apparatus 700 for simulating myocardial blood flow comprises:
a first unit 710, configured to acquire an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery;
a second unit 720, configured to, for the at least one first region, acquire a first time-density curve to obtain a maximum slope of the first time-density curve;
a third unit 730, configured to, for the at least one second region, acquire a second time-density curve to obtain a maximum enhancement value of the second time-density curve; and
a fourth unit 740, configured to determine the myocardial blood flow according to the maximum slope and the maximum enhancement value.

It should be understood that the units of the apparatus 700 shown in FIG. 7 may correspond to steps S110 and S130 to S150 in the method 100 described with reference to FIG. 1. Thus, the operations, features, and advantages described above with respect to the method 100 are also applicable to the apparatus 700 and the units comprised therein. For the sake of conciseness, some operations, features, and advantages are not described again here.

Although specific functions are discussed above with reference to specific units, it should be noted that the functions of each unit discussed herein may be divided into multiple units, and/or at least some of the functions of multiple units may be combined into a single unit. A specific unit performing an action discussed herein includes the specific unit itself performing the action, or the specific unit invoking or otherwise accessing another component or unit that performs the action (or performs the action together with the specific unit). Therefore, a specific unit that performs an action may include the specific unit that performs the action itself and/or another unit that the specific unit invokes or otherwise accesses to perform the action.

It should also be understood that the various technologies may be described in the general context of a software or hardware element or a program unit. Each of the units described with respect to FIG. 7 above may be implemented in hardware or in hardware that combines software and/or firmware. For example, these units may be implemented as computer program codes/instructions, which are configured to execute on one or more processors and stored on a computer-readable storage medium. Alternatively, these units may be implemented as hardware logic/circuitry. For example, in some aspects, one or more of the first unit 710, the second unit 720, the third unit 730, and the fourth unit 740 may be implemented together in a system on chip (SoC). The SoC may include an integrated circuit chip (which includes a processor (e.g., a central processing unit (CPU), a microcontroller, a microprocessor, a digital signal processor) DSP, etc.), a memory, one or more communication interfaces, and/or one or more components in other circuits), and may optionally execute the received program codes and/or include embedded firmware to perform functions.

According to another aspect of the present disclosure, there is provided an electronic device, comprising: at least one processor; and a memory in communicative connection with the at least one processor, wherein the memory stores instructions which are executable by the at least one processor, and the instructions are executed by the at least one processor, to enable the at least one processor to perform the method for simulating myocardial blood flow described above.

According to another aspect of the present disclosure, there is provided a non-transitory computer-readable storage medium storing a computer program, wherein the computer program is configured to cause a computer to perform the method for simulating myocardial blood flow described above.

According to another aspect of the present disclosure, there is provided a computer program product comprising a computer program which, when executed by a processor, implements the method for simulating myocardial blood flow described above.

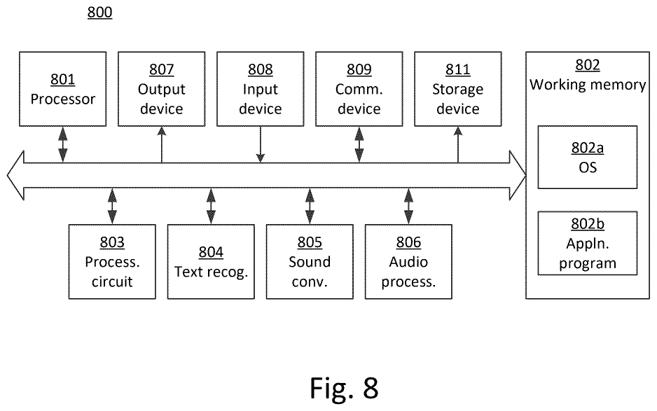

FIG. 8 is a block diagram showing an example of an electronic device 800 according to an exemplary aspect of the present disclosure. It should be noted that the structure shown in FIG. 8 is merely an example; according to a specific manner of implementation, the electronic device of the present disclosure may only comprise one or more of the constituent parts shown in FIG. 8.

The electronic device 800 may be, for example, a general-purpose computer (e.g., a laptop computer, a tablet computer, or various other types of computer), a mobile phone, or a personal digital assistant. According to some aspects, the electronic device 800 may be a cloud computing device and a smart device. According to some aspects, the electronic device 800 may be an X-ray imaging device, such as a computed tomography CT device.

According to some aspects, the electronic device 800 may be configured to process any one of images, text, and audio, and to transmit the processing results to an output device, so that it is provided to a user. For example, the output device may be a display screen or a device including a display screen, or may be a sound output device such as a headphone, a speaker, or an oscillator. For example, the electronic device 800 may be configured to perform target detection on an image and transmit the result of target detection to a display device for display; the electronic device 800 may further be configured to perform enhancement processing on the image and transmit the enhancement results to the display device for display. The electronic device 800 may further be configured to recognize text in the image and transmit the recognition results to the display device for display and/or to convert the recognition results into sound data and transmit same to a sound output device for playback. The electronic device 800 may further be configured to recognize and process audio and transmit the recognition results to the display device for display and/or convert the processing results into sound data and transmit same to the sound output device for playback.

The electronic device 800 may comprise an image processing circuit 803, and the image processing circuit 803 may be configured to perform various types of image processing on the image. The image processing circuit 803 may, for example, be configured to perform at least one of the following image processing on the image: noise reduction, normalization, registration, geometric correction, feature extraction, detection, and/or recognition of objects in the image, image enhancement, and detection and/or recognition of text included in the image, etc.

The electronic device 800 may further include a text recognition circuit 804, and the text recognition circuit 804 is configured to perform text detection and/or recognition (e.g., OCR processing) of text regions in the image, thereby obtaining text data. The text recognition circuit 804 may be implemented, for example, by a dedicated chip. The electronic device 800 may further include a sound conversion circuit 805, and the sound conversion circuit 805 is configured to convert the text data into sound data. The sound conversion circuit 805 may be implemented, for example, by a dedicated chip.

The electronic device 800 may further include an audio processing circuit 806, and the audio processing circuit 806 is configured to convert audio to text, thereby obtaining text data corresponding to the audio. The audio processing circuit 806 may further be configured to process the text data corresponding to the audio. For example, the processing may include keyword extraction, intent recognition, intelligent recommendation, intelligent question and answer, etc. The audio processing circuit 806 may be implemented, for example, by a dedicated chip. The sound conversion circuit 805 may further be configured to convert audio processing results into sound data, so as to be suitable for application scenarios such as voice assistants or virtual customer service.

One or more of the various circuits described above (e.g., the image processing circuit 803, the text recognition circuit 804, the sound conversion circuit 805, and the audio processing circuit 806) may be implemented by using custom hardware, and/or by using hardware, software, firmware, middleware, microcodes, hardware description language, or any combination thereof. For example, one or more of the various circuits described above may be implemented by using assembly language or hardware programming language (such as VERILOG, VHDL, C++) to program hardware (e.g. a programmable logic circuit including a field programmable gate array (FPGA) and/or a programmable logic array (PLA)) according to logic and algorithms of the present disclosure.

According to some manners of implementation, the electronic device 800 may further comprise an output device 807. The output device 807 may be any type of device used to present information, and may include, but is not limited to, a display screen, a terminal with a display function, earphones, a loudspeaker, a vibrator, and/or a printer, etc.

According to some manners of implementation, the electronic device 800 may further comprise an input device 808. The input device 808 may be any type of device configured to input information to the electronic device 800, and may include, but is not limited to, various types of sensors, mice, keyboards, touch screens, push buttons, control sticks, microphones, and/or remote controllers, etc.

According to some manners of implementation, the electronic device 800 may further comprise a communication device 809. The communication device 809 may be any type of device or system enabling communication with an external device and/or with a network, and may include, but is not limited to, a modem, a network card, an infrared communication device, a wireless communication device and/or a chipset, e.g. a Bluetooth device, a 802.11 device, a WiFi device, a WiMax device, a cellular communication device and/or the like.

According to some manners of implementation, the electronic device 800 may further comprise a processor 801. The processor 801 may be any type of processor, and may include, but is not limited to, one or more general-purpose processors and/or one or more dedicated processors (e.g., special processing chips). The processor 801 may, for example, be, but is not limited to, a central processing unit (CPU), a graphics processing unit (CPU), various types of dedicated artificial intelligence (AI) computing chips, or the like

The electronic device 800 may further comprise a working memory 802 and a storage device 811. The processor 801 may be configured to be able to acquire and execute computer-readable instructions stored in the working memory 802, the storage device 811, or another computer-readable medium, such as program codes of an operating system 802a, program codes of an application program 802b, etc. The working memory 802 and the storage device 811 are examples of computer-readable storage media used to store instructions, and the stored instructions can be executed by the processor 801 to implement the various functions described previously. The working memory 802 may comprise both a volatile memory and a non-volatile memory (e.g., RAM, ROM, etc.). The storage device 811 may comprise a hard disk drive, a solid state drive, and a removable medium, including external and removable drives, memory cards, flash memories, floppy disks, optical disks (e.g., CD, DVD), storage arrays, network attached storage, storage area networks, etc. The working memory 802 and the storage device 811 may both be collectively referred to as memory or a computer-readable storage medium herein, and may be non-transitory media capable of storing computer-readable, processor-executable program instructions as computer program codes, which may be executed by the processor 801 as a specific machine configured to implement the operations and functions described in the examples herein.

According to some manners of implementation, the processor 801 may control and schedule at least one of the image processing circuit 803, the text recognition circuit 804, the sound conversion circuit 805, the audio processing circuit 806, and other various apparatuses and circuits included in the electronic device 800. According to some manners of implementation, at least some of the constituent parts shown in FIG. 8 may be connected to and/or communicate with each other via a bus 810.

Software elements (programs) may be located in the working memory 802, including but not limited to an operating system 802a, one or more application programs 802b, a driver, and/or other data and codes.

According to some manners of implementation, instructions for performing control and scheduling described previously may be included in the operating system 802a or one or more application programs 802b.

According to some manners of implementation, instructions for performing the method steps described in the present disclosure may be included in one or more application programs 802b, and the various modules of the electronic device 800 described above may be implemented by the processor 801 reading and executing the instructions of the one or more application programs 802b. In other words, the electronic device 800 may comprise the processor 801 and a memory storing a program (e.g., the working memory 802 and/or the storage device 811), the program comprising instructions which, when executed by the processor 801, cause the processor 801 to perform the method as described in the various aspects of the present disclosure.

According to some manners of implementation, some or all of the operations performed by at least one of the image processing circuit 803, the text recognition circuit 804, the sound conversion circuit 805, and the audio processing circuit 807 may be implemented by the processor 801 reading and executing instructions of one or more application programs 802b.

Executable codes or source codes of instructions of software elements (programs) may be stored in a non-transitory computer-readable storage medium (e.g., the storage device 811), and when executed, may be stored in the working memory 802 (possibly compiled and/or installed). Therefore, the present disclosure provides a computer-readable storage medium storing a program, the program comprising instructions which, when executed by a processor of an electronic device, cause the electronic device to perform the method as described in the various aspects of the present disclosure. According to another manner of implementation, executable codes or source codes of instructions of software elements (programs) may also be downloaded from a remote location.

It should also be understood that various modifications may be made according to specific requirements. For example, various circuits, units, modules, or elements may be implemented by using custom hardware, and/or by using hardware, software, firmware, middleware, microcodes, hardware description languages, or any combination thereof. For example, some or all of the circuits, units, modules or elements included in the disclosed methods and devices may be implemented by using assembly language or hardware programming language (such as VERILOG, VHDL, C++) to program hardware (e.g. a programmable logic circuit including a field programmable gate array (FPGA) and/or a programmable logic array (PLA)) according to logic and algorithms of the present disclosure.

According to some manners of implementation, the processor 801 in the electronic device 800 may be distributed on a network. For example, some processing may be performed by one processor, while other processing may be performed by another processor remote from the one processor. Other modules of the electronic device 800 may also be similarly distributed. Thus, the electronic device 800 may be interpreted as being a distributed computing system that performs processing at multiple locations. The processor 801 of the electronic device 800 may also be a processor of a cloud computing system, or a processor in which a blockchain is incorporated.

Although the aspects or examples of the present disclosure have been described with reference to the drawings, it should be understood that the methods, systems, and devices described above are merely exemplary aspects or examples, and the scope of the present disclosure is not limited by these aspects or examples, but is defined solely by the granted claims and the equivalent scope thereof. Various key elements in the aspects or examples may be omitted or may be replaced by equivalent key elements thereof. In addition, the steps may be executed in an order different from that described in the present disclosure. Further, various key elements in the aspects or examples may be combined in various ways. Importantly, as technology evolves, many key elements described here may be replaced by equivalent key elements appearing after the present disclosure.

1. A method for simulating myocardial blood flow, comprising:
acquiring an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery;
for the at least one first region, acquiring a first time-density curve to obtain a maximum slope of the first time-density curve;
for the at least one second region, acquiring a second time-density curve to determine a maximum enhancement value of the second time-density curve; and
determining the myocardial blood flow according to the maximum slope and the maximum enhancement value.

2. The method as claimed in claim 1, wherein the maximum slope is determined by:
for a plurality of first subsegments of the first time-density curve, calculating a first slope of each first subsegment; and
determining a maximum slope calculated in the plurality of first subsegments to be the maximum slope,
wherein the plurality of first subsegments are composed of lines connecting each two temporally adjacent first monitored CT values among a plurality of first monitored CT values of the at least one first region in an angiography process.

3. The method as claimed in claim 1, wherein the maximum enhancement value is determined by:
calculating a difference between maximum and minimum values among a plurality of second monitored CT values of the at least one second region in an angiography process and taking same as the maximum enhancement value.

4. The method as claimed in claim 1, wherein determining the myocardial blood flow according to the maximum slope and the maximum enhancement value comprises:
calculating the myocardial blood flow using a weighted linear combination of the maximum slope and the maximum enhancement value.

5. The method as claimed in claim 1, wherein acquiring the angiographic image including the coronary artery and the left ventricle adjacent to the coronary artery further comprises:
preprocessing the angiographic image to obtain a preprocessed angiographic image;
dividing the preprocessed angiographic image into the myocardial region of the left ventricle and the lumen region of the coronary artery;
in the myocardial region of the left ventricle, extracting at least one first region of interest and taking same as the at least one first region; and
in the lumen region of the coronary artery, extracting at least one second region of interest and taking same as the at least one second region.

6. The method as claimed in claim 1, wherein:
the acquisition of the first time-density curve starts from a first trigger time, and the first trigger time is a time at which a first monitored CT value of the at least one first region exceeds a preset first trigger value for the first time; and
the acquisition of the second time-density curve starts from a second trigger time, and the second trigger time is a time at which a second monitored CT value of the at least one second region exceeds a preset second trigger value for the first time.

7. The method as claimed in claim 6, wherein:
the acquisition of the first time-density curve stops at a first cutoff time, and the first cutoff time is configured to be a time at which the first monitored CT value of the at least one first region exceeds a preset first threshold value for the first time; and
the acquisition of the second time-density curve stops from a second cutoff time, and the second cutoff time is configured to be a time at which the second monitored CT value of the at least one second region exceeds a preset second threshold value for the first time.

8. An apparatus for simulating myocardial blood flow, the apparatus comprising:
a first unit, configured to acquire an angiographic image including a coronary artery and a left ventricle adjacent to the coronary artery, the angiographic image comprising at least one first region and at least one second region, wherein the at least one first region is located in a myocardial region of the left ventricle, and the at least one second region is located in a lumen region of the coronary artery;
a second unit, configured to, for the at least one first region, acquire a first time-density curve to obtain a maximum slope of the first time-density curve;
a third unit, configured to, for the at least one second region, acquire a second time-density curve to obtain a maximum enhancement value of the second time-density curve; and
a fourth unit, configured to determine the myocardial blood flow according to the maximum slope and the maximum enhancement value.

9. An electronic device, comprising:
at least one processor; and
at least one non-transitory memory in communicative connection with the at least one processor, wherein the at least one non-transitory memory stores instructions which are executable by the at least one processor, and the instructions are executed by the at least one processor to enable the at least one processor to perform the method as claimed in claim 1.
